(19) United States
(12) Patent Application Publication
(10) Pub. No.: US 2023/0371868 A1
(43) Pub. Date: Nov. 23, 2023

(54) Offset Fluid Path for Blood Draw Device

(71) Applicant: Becton, Dickinson and Company, Franklin Lakes, NJ (US)

(72) Inventors: Megan S. Scherich, Salt Lake City, UT (US); Weston F. Harding, Lehi, UT (US); Evan Barbour VandenBrink, San Mateo, CA (US); John M. Lackey, West Valley City, UT (US); Serena Robinson Agrawal, Salt Lake City, UT (US)

(21) Appl. No.: 18/320,254

(22) Filed: May 19, 2023

Related U.S. Application Data
(60) Provisional application No. 63/344,242, filed on May 20, 2022.

(51) Int. Cl. A61B 5/15

(52) U.S. Cl. CPC A61B 5/150992; A61B 5/15003; A61M 25/06

(57) ABSTRACT

A blood draw device for use with a peripheral intravenous catheter (PIVC) includes a catheter having a proximal end, a distal end, and a sidewall therebetween defining a lumen, an introducer having a proximal end, a distal end, and a sidewall therebetween defining an inner volume configured to movably receive the catheter, with the introducer defining a longitudinal axis, and an actuator movably coupled to the introducer and connected to the catheter. The actuator having a first portion disposed outside of the introducer and a second portion disposed in the inner volume of the introducer, with the catheter having a first portion extending parallel to the longitudinal axis of the introducer and a second portion extending oblique to the longitudinal axis of the introducer.

### CROSS-REFERENCE TO RELATED APPLICATION

The present application claims priority to U.S. Provisional Application Ser. No. 63/344,242 entitled “Offset Fluid Path for Blood Draw Device” filed May 20, 2022, the entire disclosure of which is hereby incorporated by reference in its entirety.

### BACKGROUND OF THE INVENTION

### Field of the Invention

The present disclosure relates generally to an offset fluid path for a blood draw device.

### Description of Related Art

Blood collection devices, when used with indwelling IV catheters, can include displaceable catheters that are advanced beyond the tip of the indwelling catheter for blood collection. Often, when the displaceable catheter is advanced, it can encounter an obstruction, resulting in deflection of the catheter. Examples of obstructions include the friction of the seal within the blood collection device, torturous path within an integrated catheter, pinching of the catheter tubing as it dives into the skin, thrombus, fibrin, and valves. Such deflections can reduce the ability of the displaceable catheter to extend beyond the tip of the indwelling catheter, and thus limits their use for blood collection.

### SUMMARY OF THE INVENTION

In one aspect or embodiment, a blood draw device for use with a peripheral intravenous catheter (PIVC) includes a catheter having a proximal end, a distal end, and a sidewall therebetween defining a lumen, an introducer having a proximal end, a distal end, and a sidewall therebetween defining an inner volume configured to movably receive the catheter, with the introducer defining a longitudinal axis, and an actuator movably coupled to the introducer and connected to the catheter. The actuator has a first portion disposed outside of the introducer and a second portion disposed in the inner volume of the introducer. The catheter has a first portion extending parallel to the longitudinal axis of the introducer and a second portion extending oblique to the longitudinal axis of the introducer.

The actuator is configured to move relative to the introducer to move the catheter between a first position, in which the catheter is disposed within the introducer, and a second position, in which the distal end of the catheter is disposed beyond the distal end of the introducer. The blood draw device may further include secondary catheter having a proximal end, a distal end, and a sidewall therebetween defining a lumen, the secondary catheter connected to the actuator. The proximal end of the secondary catheter may include a coupler.

The actuator may include a main body and an extension portion, with the main body of the actuator extending parallel to the longitudinal axis of the introducer, and with the extension portion of the actuator receiving the catheter and extending oblique to the longitudinal axis of the introducer.

The introducer may define a groove extending in a direction extending from the proximal end of the introducer to the distal end of the introducer, with the groove positioned within the inner volume of the introducer and configured to receive a portion of the catheter during use of the blood draw device. The groove may be U-shaped, V-shaped, and/or squared-shaped.

In one aspect or embodiment, a blood draw device for use with a peripheral intravenous catheter (PIVC) includes a catheter having a proximal end, a distal end, and a sidewall therebetween defining a lumen, a secondary catheter having a proximal end, a distal end, and a sidewall therebetween defining a lumen, an introducer having a proximal end, a distal end, and a sidewall therebetween defining an inner volume configured to movably receive the catheter, with the introducer defining a longitudinal axis, and an actuator movably coupled to the introducer. The actuator has a first portion disposed outside of the introducer and a second portion disposed in the inner volume of the introducer. The actuator includes a catheter connection connected to the catheter and a secondary catheter connection connected to the secondary catheter. The catheter and the secondary catheter each extend parallel to the longitudinal axis of the introducer, with the catheter offset a distance from the secondary catheter.

The catheter connection may be offset from the secondary catheter connection via an angled body portion.

The actuator may include a main body having a top surface and a bottom surface positioned opposite from the top surface, with the catheter connection extending from the bottom surface of the main body of the actuator. The secondary catheter connection may extend between the top surface and the bottom surface of the main body of the actuator.

In one aspect or embodiment, a method of forming a catheter for a blood draw device for use with a peripheral intravenous catheter (PIVC) comprising a catheter defining a lumen, an introducer defining an inner volume configured to movably receive the catheter, the introducer defining a longitudinal axis, and an actuator movably coupled to the introducer and connected to the catheter, with the method including molding the catheter to include a first portion extending parallel to the longitudinal axis of the introducer and a second portion extending oblique to the longitudinal axis of the introducer.

Molding the catheter may include a curved core pin pull. Molding the catheter may include bending the second portion of the catheter relative to the first portion of the catheter.

### BRIEF DESCRIPTION OF THE DRAWINGS

FIG. 1 is a schematic illustration of a blood draw device in a first configuration in accordance with an aspect of the present disclosure;

FIG. 2 is a schematic illustration of the blood draw device of FIG. 1 in a second configuration in accordance with an aspect of the present disclosure;

FIG. 3 is a perspective view of a blood draw device in accordance with an aspect of the present disclosure;

FIG. 4 is an exploded perspective view of the blood draw device of FIG. 3;

FIG. 5 is a partial rear view of the blood draw device of FIG. 3;

FIG. 6 is a side view of the blood draw device of FIG. 3;

FIG. 7 is a cross-sectional view of a prior art blood draw device;

FIG. 8 is an enlarged cross-sectional view of Area A shown in FIG. 7;

FIG. 9 is a cross-sectional view of a prior art blood draw device, showing a catheter with a deflected position;

FIG. 10 is a partial side view of a blood draw device according to one aspect or embodiment of the present disclosure;

FIG. 11 is a partial perspective view of the blood draw device of FIG. 10;

FIG. 12 a partial cross-sectional view of a blood draw device according to one aspect or embodiment of the present disclosure;

FIG. 13 a partial cross-sectional view of a blood draw device according to one aspect or embodiment of the present disclosure;

FIG. 14A is a partial cross-sectional view of pusher mold according to one aspect or embodiment of the present disclosure;

FIG. 14B is a partial top view of the pusher mold of FIG. 14A;

FIG. 15 is a partial cross-sectional view of pusher mold according to one aspect or embodiment of the present disclosure;

FIG. 16A is a partial cross-sectional view of a blood draw device according to one aspect or embodiment of the present disclosure;

FIG. 16B is a front view of the blood draw device of FIG. 16A;

FIG. 17 is a partial cross-sectional view of a blood draw device according to one aspect or embodiment of the present disclosure;

FIG. 18 is a partial cross-sectional view of a blood draw device according to one aspect or embodiment of the present disclosure;

FIG. 19 is a partial top view of a blood draw device according to one aspect or embodiment of the present disclosure, showing a groove in an introducer; and

FIG. 20 is a partial side view of the blood draw device of FIG. 19.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

Any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

U.S. Pat. No. 11,090,461, which discloses medical devices including blood draw devices, is incorporated by reference herein in its entirety.

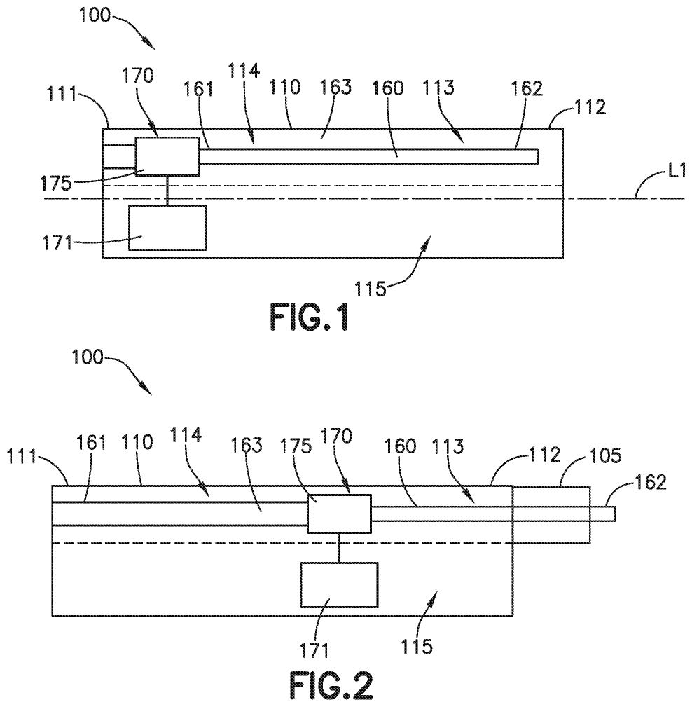

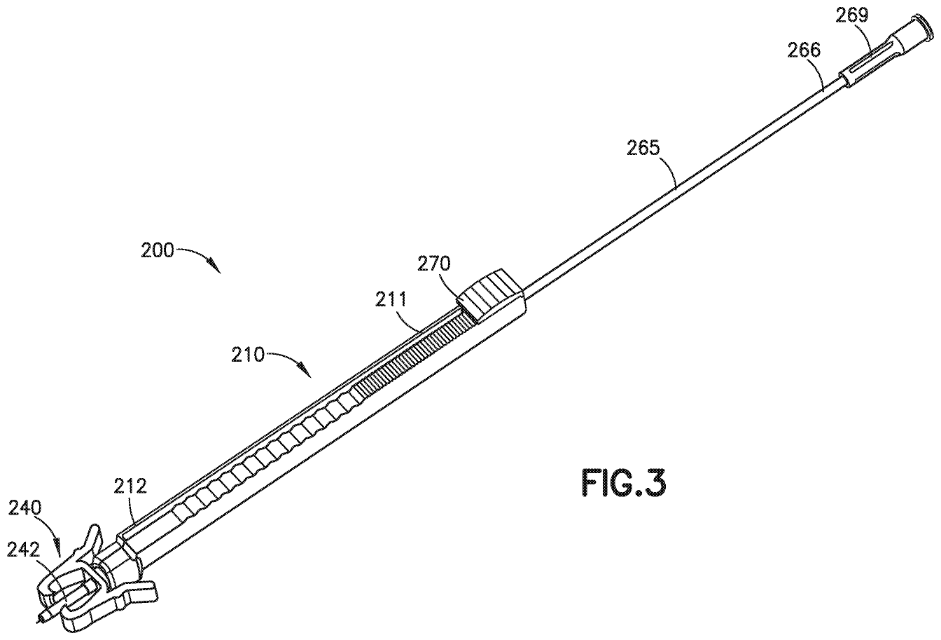

Referring to FIGS. 1 and 2, in one aspect or embodiment, a blood draw device 100 for phlebotomy through a peripheral intravenous line or catheter in a first configuration and second configuration, respectively, is shown. The blood draw device 100 (also referred to herein as “blood draw device” or “transfer device”) can be any suitable shape, size, and/or configuration. As described in further detail herein, the transfer device 100 is configured to couple to and/or otherwise engage an indwelling peripheral intravenous catheter (PIVC) 105 to transfer fluid from (e.g., aspiration of blood) and/or transfer fluid to (e.g., infusion of a drug or substance) a portion of a patient.

The transfer device 100 includes at least an introducer 110, a catheter 160 (or cannula), and an actuator 170. The introducer 110 can be any suitable configuration. For example, in some embodiments, the introducer 110 can be an elongate member having a substantially circular cross-sectional shape. In some embodiments, the shape of the introducer 110 and/or one or more features or surface finishes of at least an outer surface of the introducer 110 can be arranged to increase the ergonomics of the transfer device 100, which in some instances, can allow a user to manipulate the transfer device 100 with one hand (i.e., single-handed use). The introducer 110 defines a longitudinal axis L1.

The introducer 110 has a proximal end portion 111 and a distal end portion 112 and defines an inner volume 113. Although not shown in FIGS. 1 and 2, the proximal end portion 111 of the introducer 110 can include an opening or port configured to movably receive a portion of the catheter 160. As such, a first portion of the catheter 160 can be disposed within the inner volume 113 and a second portion of the catheter 160 can be disposed outside of the inner volume 113. The opening or port can be any suitable configuration. For example, in some embodiments, the opening and/or port can include a seal or the like configured to form a substantially fluid tight seal with an outer surface of the portion of the catheter 160 disposed therein. In other embodiments, the arrangement of the opening and/or port can be such that a user can place the catheter 160 in selective contact with a surface of the proximal end portion 111 defining the opening and/or port, which in turn, can clamp and/or pinch the catheter 160 to selectively obstruct a lumen of the catheter 160, as described in further detail herein with reference to specific embodiments.

The distal end portion 112 of the introducer 110 includes and/or is coupled to a lock configured to physically and fluidically couple the introducer 110 to the PIVC 105 (see e.g., FIG. 2). For example, in some embodiments, the distal end portion 112 can include a coupler or the like such as a Luer Lok™ configured to physically and fluidically couple to an associated coupler of the lock. In some embodiments, the lock is configured to selectively engage and/or contact the PIVC 105 to couple the introducer 110 thereto. For example, in some embodiments, the shape, size, and/or arrangement of the lock is such that the lock forms three points of contact with the PIVC 105. In some embodiments, such an arrangement can provide structural rigidity and/or support to the PIVC 105 as a portion of the lock (e.g., a blunt tip cannula or the like) is inserted into a portion of the PIVC 105, as described in further detail herein.

In some aspects or embodiments, the distal end portion 112 of the introducer 110 can include and/or can be coupled to a support member or the like that is operable in placing the introducer 110 and/or transfer device 100 at a predetermined angle relative to a target surface. For example, in some embodiments, the arrangement of the lock can be such that placing a predetermined portion of the lock in contact with a target surface, in turn, places the introducer 110 and/or transfer device 100 at a predetermined and/or desired angle relative to the target surface. In other embodiments, a support member and/or the like can be coupled to the distal end portion 112 of the introducer 110 and configured to place the introducer 110 and/or transfer device 100 at the predetermined and/or desired angle relative to the target surface. In some instances, the target surface can be a cutaneous surface of a body through which the PIVC 105 is inserted (e.g., an outer surface of a patient's arm or the like). In some aspects or embodiments, the predetermined angle can be, for example, between about 0° and about 30°, between about 4° and about 15°, between about 8° and about 10°, or any other suitable angle.

In some aspects or embodiments, the distal end portion 112 of the introducer 110 (and/or the lock) can include a seal or the like that can be transferred from a sealed configuration to a substantially open configuration to place at least a portion of the inner volume 113 in fluid communication with the lock. The seal can include a back flow prevention mechanism such as a one-way valve or the like that can allow, for example, the catheter 160 to be advanced in the distal direction therethrough while limiting and/or substantially preventing a fluid flow, outside the catheter 160, in the proximal direction through the seal.

As described above, the introducer 110 defines the inner volume 113, which extends between the proximal end portion 111 and the distal end portion 112. The inner volume 113 has and/or defines a first portion 114 configured to receive a first portion 171 of the actuator 170 and a second portion 115 configured to receive the catheter 160 and a second portion 175 of the actuator 170, as shown in FIGS. 1 and 2. More specifically, an inner surface of the introducer 110 that defines the inner volume 113 can have, for example, a tortuous cross-sectional shape (not shown in FIGS. 1 and 2) such that an axis defined by the first portion 114 of the inner volume 113 is parallel to and offset from an axis defined by the second portion 115 of the inner volume 113. In this manner, the first portion 114 of the inner volume 113 can be spaced apart from the second portion 115 of the inner volume 113 without being fluidically isolated therefrom. The first portion 114 of the inner volume 113 can extend through a wall of the introducer 110. In other words, the introducer 110 can define a slot, channel, track, opening, and/or the like that is in fluid communication with the first portion 114 of the inner volume 113. Conversely, the second portion 115 of the inner volume 113 can be entirely defined and/or enclosed (at least in the circumferential direction) by the introducer 110. Moreover, in some aspects or embodiments, the tortuous cross-sectional shape of the inner volume 113 is such that the second portion 115 cannot be viewed (e.g., is out of the line of sight) via the slot or the like in fluid communication with the first portion 114 of the inner volume 113, which in turn, can limit and/or substantially prevent contamination of the catheter 160 disposed therein.

The catheter 160 of the transfer device 100 includes a proximal end portion 161 and a distal end portion 162 and defines a lumen 163 that extends through the proximal end portion 161 and the distal end portion 162. The catheter 160 is movably disposed within the second portion 115 of the inner volume 113 defined by the introducer 110 and is coupled to the actuator 170. The catheter 160 can be moved (e.g., via movement of the actuator 170) between a first position and a second position to transition the transfer device 100 between the first configuration and the second configuration, respectively. More specifically, at least the distal end portion 162 of the catheter 160 is disposed within the second portion 115 of the inner volume 113 when the catheter 160 is in the first position (FIG. 1) and at least a portion of the catheter 160 extends through the PIVC 105 to place a distal end of the catheter 160 in a distal position relative to a portion of the PIVC 105 when the catheter 160 is in the second position (FIG. 2). Although not shown in FIGS. 1 and 2, in some embodiments, the transfer device 100 can include a secondary catheter or the like that is coupled to the actuator 170 and in fluid communication with the catheter 160. In such embodiments, the secondary catheter can be, for example, disposed in a proximal position relative to the catheter 160 and can be configured to extend through the opening and/or port defined by the proximal end portion 111 of the introducer 110. In this manner, a proximal end portion of the secondary catheter can be coupled to a vacuum (air or liquid) source, a fluid reservoir, fluid source, syringe, and/or the like, which in turn, places the catheter 160 in fluid communication therewith. Moreover, in embodiments including the secondary catheter, the catheter 160 can be entirely disposed within the introducer 110 when the catheter 160 is in the first position.

The catheter 160 can be any suitable shape, size, and/or configuration. For example, in some embodiments, at least a portion of the catheter 160 can have an outer diameter (e.g., between a 10-gauge and a 30-gauge) that is substantially similar to or slightly smaller than an inner diameter defined by a portion of the lock coupled to the distal end portion 112 of the introducer 110. In this manner, an inner surface of the portion of the lock can guide the catheter 160 as the catheter 160 is moved between the first position and the second position. Such an arrangement can limit and/or can substantially prevent bending, deforming, and/or kinking of a portion of the catheter 160 as the portion is moved between the first position and the second position. The catheter 160 can have a length that is sufficient to place a distal surface of the catheter 160 in a desired position relative to a distal surface of the PIVC 105 when the catheter 160 is in the second position. In other words, the length of the catheter 160 can be sufficient to define a predetermined and/or desired distance between the distal surface of the catheter 160 and the distal surface of the PIVC 105 when the catheter 160 is in the second position. In some instances, placing the distal surface of the catheter 160 at the predetermined and/or desired distance from the distal surface of the PIVC 105 can, for example, place the distal surface of the catheter 160 in a desired position within a vein, as described in further detail herein.

The catheter 160 can be formed from any suitable material or combination of materials, which in turn, can result in the catheter 160 having any suitable stiffness or durometer. In some embodiments, at least a portion of the catheter 160 can be formed of a braided material or the like, which can change, modify, and/or alter a flexibility of the catheter 160 in response to a bending force or the like. In some aspects or embodiments, forming the catheter 160 of the braided material or the like can reduce a likelihood of kinking and/or otherwise deforming in an undesired manner. In addition, forming at least a portion of the catheter 160 of a braided material can result in a compression and/or deformation in response to a compression force exerted in a direction of a longitudinal centerline defined by the catheter 160 (e.g., an axial force or the like). In this manner, the catheter 160 can absorb a portion of force associated with, for example, impacting an obstruction or the like. As described in further detail herein, in some instances, at least a portion of the catheter 160 can deform in response to the force associated with impacting such an obstruction or the like.

The actuator 170 of the transfer device 100 can be any suitable shape, size, and/or configuration. As described above, the actuator 170 includes the first portion 171 movably disposed within the first portion 114 of the inner volume 113 and the second portion 175 movably disposed within the second portion 115 of the inner volume 113 and coupled to the catheter 160. Although not shown in FIGS. 1 and 2, the actuator 170 can have a cross-sectional shape that is associated with and/or otherwise corresponds to the cross-sectional shape of the inner volume 113 (e.g., the tortuous cross-sectional shape). Thus, an axis defined by the first portion 171 of the actuator 170 is parallel to and offset from an axis defined by the second portion 175 of the actuator 170.

The arrangement of the actuator 170 and the introducer 110 is such that the first portion 171 extends through the slot or the like in fluid communication with the first portion 114 of the inner volume 113. As such, a first region of the first portion 171 of the actuator 170 is disposed outside of the introducer 110 and a second region of the first portion 171 of the actuator 170 is disposed in the first portion 114 of the inner volume 113. In this manner, a user can engage the first region of the first portion 171 of the actuator 170 and can move the actuator 170 relative to the introducer 110 to move the catheter 160 coupled to the second portion 175 of the actuator 170 between the first position and the second position. Although not shown in FIGS. 1 and 2, in some embodiments, the first portion 171 of the actuator 170 can include a tab, protrusion, and/or surface that is in contact with an outer surface of the introducer 110. In such embodiments, the outer surface of the introducer 110 can include, for example, a set of ribs, ridges, bumps, grooves, and/or the like along which the tab, protrusion, and/or surface of the first portion 171 advances when the actuator 170 is moved relative to the introducer 110, which in turn, produces a haptic output or feedback (acoustic, tactile and visual) which can provide an indication associated with a position of the distal end portion 162 of the catheter 160 to the user.

In some aspects or embodiments, the arrangement of the first portion 171 of the actuator 170 and the outer surface of the introducer 110 is such that the actuator 170 is disposed at an angle relative to the introducer 110. That is to say, the contact between the first portion 171 of the actuator 170 and the outer surface of the introducer 110 tilts the actuator 170 relative to the introducer 110. Accordingly, a longitudinal centerline of the actuator 170 can be nonparallel to a longitudinal centerline of the introducer 110. Furthermore, with the actuator 170 coupled to the proximal end portion 161 of the catheter 160, angling and/or tilting the actuator 170 results in a force (e.g., a pre-load force or the like) exerted on the catheter 160 that is sufficient to bend at least a portion of the catheter 160 (e.g., the catheter 160 is placed in a biased configuration), as described in further detail herein.

In some aspects or embodiments, the transfer device 100 can be disposed in the first configuration prior to use (e.g., shipped, stored, prepared, etc. in the first configuration). In use, a user can manipulate the transfer device 100 to couple the introducer 110 to the indwelling PIVC 105 (e.g., via the lock coupled to and/or assembled with the introducer 110). With the transfer device 100 coupled to the PIVC 105, the user can engage the first portion 171 of the actuator 170 to move the actuator 170 relative to the introducer 110, which in turn, moves the catheter 160 from the first position (e.g., disposed within the introducer 110) toward the second position. The arrangement of the actuator 170 and the introducer 110 is such that advancing the actuator 170 relative to the introducer 110 produces a haptic output and/or feedback configured to provide an indicator associated with the position of the distal end portion 162 of the catheter 160 relative to the introducer 110 and/or the PIVC 105 to the user. For example, based on the haptic feedback or any other suitable indicator, the user can place the catheter 160 in the second position such that the distal surface of the catheter 160 extends a desired distance beyond the distal surface of the PIVC 105, as described above.

With the catheter 160 in the second position (e.g., with the transfer device 100 in the second configuration shown in FIG. 2), the user can establish fluid communication between a fluid reservoir, fluid source, syringe, and/or the like, and the catheter 160. For example, as described above, in some embodiments, the user can couple the secondary catheter (not shown) to the fluid reservoir, fluid source, syringe, and/or the like. Although described as establishing fluid communication between the catheter 160 and the fluid reservoir or fluid source after placing the catheter 160 in the second position, in other embodiments, the user can establish fluid communication between the catheter 160 and the fluid reservoir or fluid source prior to moving the actuator 170 relative to the introducer 110. With the catheter 160 in fluid communication with the fluid reservoir and/or fluid source, the transfer device 100 can then transfer a fluid from the patient or transfer a fluid to the patient via the catheter 160 extending through and beyond the PIVC 105.

In some instances, the catheter 160 can impact an obstruction or the like as the user advances the catheter 160 (via the actuator 170) from the first position to the second position. In some such instances, the catheter 160 can be configured to bend, deform, and/or otherwise reconfigure in response to a force exerted by the user. That is to say, a force (e.g., an activation or actuation force) exerted by the user on the actuator 170 that otherwise is sufficient to move the catheter 160 toward the second position results in a deflection, deformation and/or reconfiguration of at least a portion of the catheter 160 when the catheter 160 impacts an obstruction or the like. Moreover, with at least a portion of the catheter 160 being pre-loaded (e.g., bent, bowed, biased, deflected, and/or deformed in response to the angle of the actuator 170, as described above), the deflection, deformation, and/or reconfiguration of the portion of the catheter 160 can be predetermined, anticipated, and/or the like.

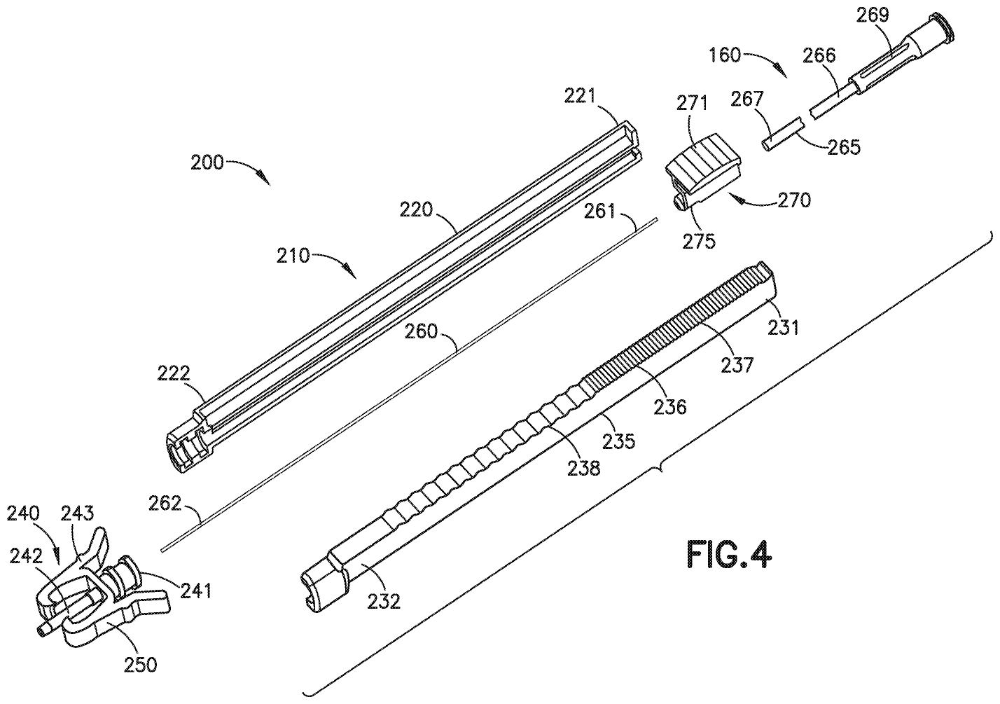

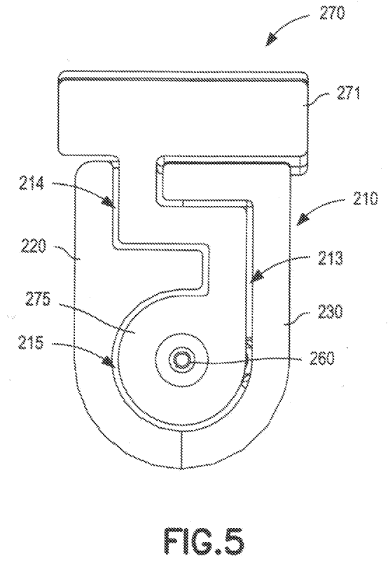

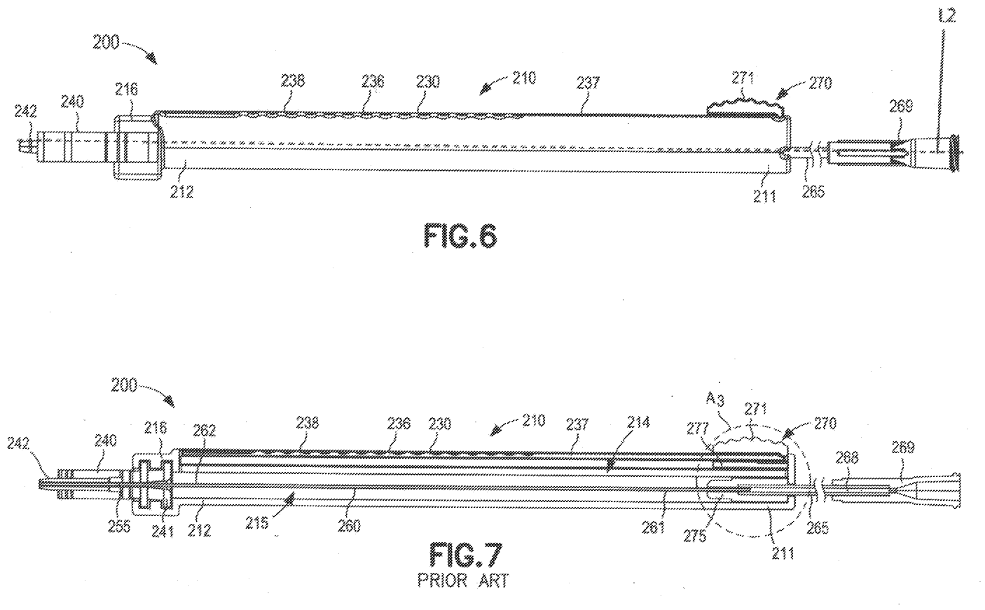

Referring to FIGS. 3-9, a blood draw device 200 according to a further embodiment is shown. The blood draw device 200 (also referred to herein as “blood draw device” or “transfer device”) can be any suitable shape, size, or configuration and can be coupled to a PIVC (not shown in FIGS. 3-9), for example, via a lock and/or adapter. As described in further detail herein, a user can transition the transfer device 200 from a first configuration to a second configuration to advance a catheter through an existing, placed, and/or indwelling PIVC (i.e., when the transfer device 200 is coupled thereto) such that at least an end portion of the catheter is disposed in a distal position relative to the PIVC. Moreover, with peripheral intravenous lines each having a shape, size, and/or configuration that can vary based on, for example, a manufacturer of the PIVC and/or its intended usage, the transfer device 200 can be arranged to allow the transfer device 200 to be coupled to a PIVC having any suitable configuration and subsequently, to advance at least a portion of a catheter through the PIVC substantially without kinking, snagging, breaking, and/or otherwise reconfiguring the catheter in an undesirable manner. In addition, the transfer device 200 can be manipulated by a user to place a distal surface of the catheter a predetermined and/or desired distance beyond a distal surface of the PIVC to be disposed within a portion of a vein that receives a substantially unobstructed flow of blood.

Referring to FIGS. 3-5, the transfer device 200 includes an introducer 210, a lock 240, a catheter 260, a secondary catheter 265, and an actuator 270. The introducer 210 can be any suitable shape, size, or configuration. For example, in some embodiments, the introducer 210 can be an elongate member having a substantially circular cross-sectional shape. In some embodiments, the shape of the introducer 210 and/or one or more features or surface finishes of at least an outer surface of the introducer 210 can be arranged to increase the ergonomics of the transfer device 200, which in some instances, can allow a user to manipulate the transfer device 200 with one hand (i.e., single-handed use). The introducer 210 defines a longitudinal axis L2.

Referring to FIGS. 3-9, the introducer 210 of the transfer device 200 includes a first member 220 and a second member 230 that are coupled to collectively form the introducer 210. The first member 220 includes a proximal end portion 221 and a distal end portion 222. The actuator 270 includes a first portion 271, a second portion 275, and a wall 277 extending therebetween. The first portion 271 of the actuator 270 is at least partially disposed within the first portion 214 of the inner volume 213 defined by the introducer 210 and the second portion 275 of the actuator 270 is disposed within a second portion 215 of the inner volume 213. The first portion 271 of the actuator 270 can be engaged and/or manipulated by a user (e.g., by a finger or thumb of the user) to move the actuator 270 relative to the introducer 210. The first portion 271 of the actuator 270 may include a set of ridges and/or any suitable surface finish that can, for example, increase the ergonomics of the actuator 270 and/or transfer device 200. The first portion 271 of the actuator 270 includes a tab 273 disposed at or near a proximal end portion of the engagement member 272. The tab 273 can be any suitable tab, rail, ridge, bump, protrusion, knob, roller, slider, etc. The tab 273 is configured to selectively engage ribs 237 on an outer surface of the second member 230 of the introducer 210. A proximal end portion 211 of the introducer 210 defines an opening 217.

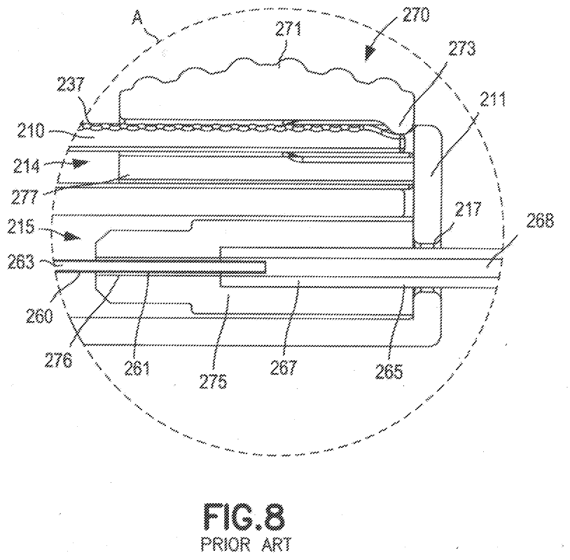

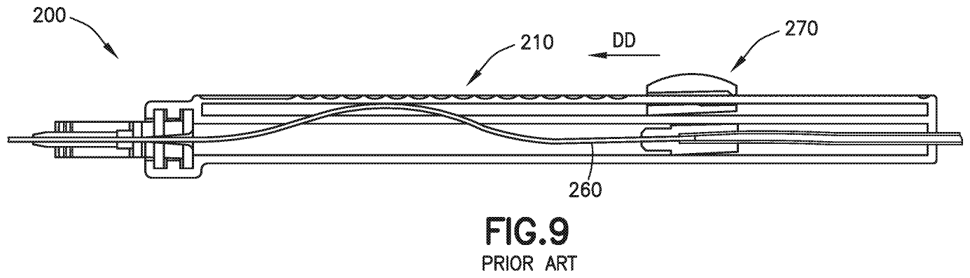

Referring to FIGS. 7-9, conventionally, the second portion 275 defines an opening 276 configured to receive a proximal end portion 261 of the catheter 260 and a distal end portion 267 of the secondary catheter 265. A portion of the secondary catheter 265 is disposed in the opening 217 defined by the introducer such that the distal end portion 267 is at least partially disposed in the second portion 215 of the inner volume 213 and coupled to the second portion 275 of the actuator 270 while the proximal end portion 266 of the secondary catheter 265 is disposed outside of the introducer 210. The proximal end portion 266 of the secondary catheter 265 is coupled to and/or otherwise includes a coupler 269. The coupler 269 is configured to physically and fluidically couple the secondary catheter 265 to any suitable device such as, for example, a fluid reservoir, fluid source, syringe, evacuated container holder (e.g., having a sheathed needle or configured to be coupled to a sheathed needle), pump, and/or the like. Positioning the distal end portion 267 of the secondary catheter 265 within the opening 276 of the actuator 270 limits a minimum height or diameter of the second portion 275 of the actuator 270. For example, in the conventional arrangement of the actuator 270 of FIGS. 7 and 8, the actuator may be overmolded onto the secondary catheter 265, which necessitates a predetermined wall thickness of material of the actuator 270 surrounding the secondary catheter 265. As shown in FIG. 9, a healthcare worker can exert a force on the actuator 270 to move the actuator 270 in a distal direction relative to the introducer 210, which in turn, moves the catheter 260 toward its second position (e.g., the distal position), as indicated by the arrow DD. In some instances, however, the catheter 260 (e.g., the distal end of the catheter 260) may encounter or impact an obstruction or the like that hinders or prevents further distal movement of the catheter 260. With the distal end of the catheter 260 in contact with the obstruction, the force exerted by the healthcare worker on the actuator 270 (e.g., in the DD direction) results in a “clutching” (e.g., deflection, deformation, bending, bowing, etc.) of the catheter 260.

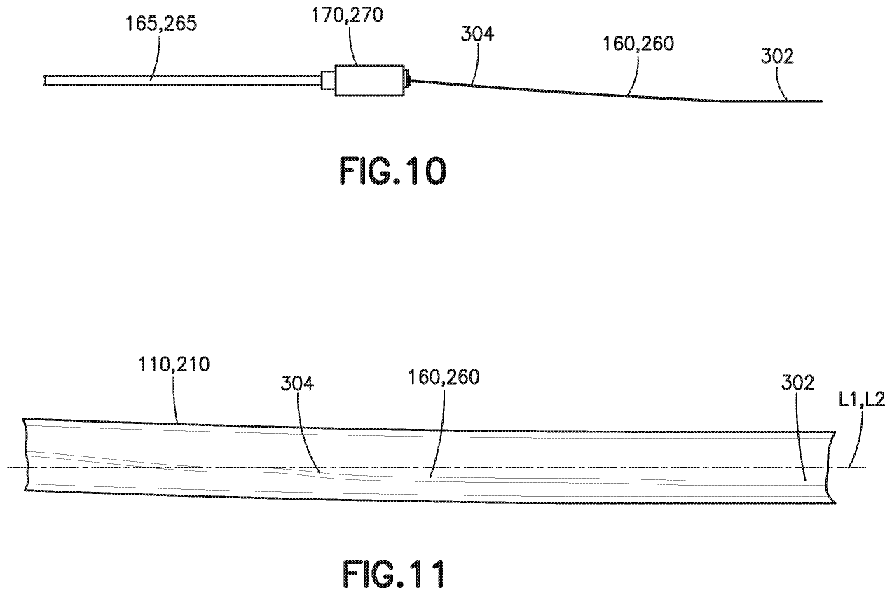

Referring to FIGS. 10 and 11, in one aspect or embodiment of the present application, the catheter 160, 260 of either device 100 or device 200, has a first portion 302 extending parallel to the longitudinal axis L1, L2 of the introducer 110, 210 and a second portion 304 extending oblique to the longitudinal axis L1, L2 of the introducer 110, 210. The second portion 304 of the catheter 160, 260 is configured to position the catheter 160, 260 closer to the introducer 110, 210 within the inner volume 113, 213 such that the catheter 160, 260 is supported by the introducer 110, 210 during use of the device 100, 200 thereby minimizing bowing and/or kinking of the catheter 160, 260.

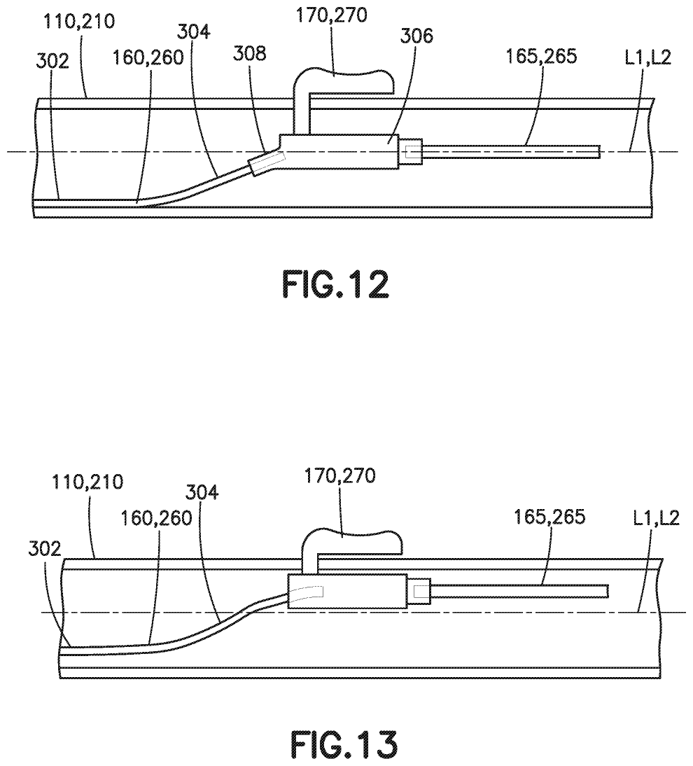

Referring to FIG. 12, in one aspect or embodiment, the actuator 170, 270 includes a main body 306 and an extension portion 308, with the main body 306 of the actuator 170, 270 extends parallel to the longitudinal axis L1, L2 of the introducer 110, 210. The extension portion 308 of the actuator 170, 270 receives the catheter 160, 260 and extends oblique to the longitudinal axis L1, L2 of the introducer 110, 210. As shown in FIG. 12, the secondary catheter 165, 265 extends from the main body 306 parallel to the longitudinal axis L1, L2 of the introducer 110, 210. The second portion 304 of the catheter 160, 260 extends from the extension portion 308. In one aspect or embodiment, the extension portion 308 extends at an angle of from 2 to 45 degrees relative to the longitudinal axis L1, L2 of the introducer 110, 210. In another aspect, the extension portion 308 extends at an angle of from 5 to 20 degrees relative to the longitudinal axis L1, L2 of the introducer 110, 210. The catheter 160, 260 may be received within the extension portion 308 or position over the extension portion 308.

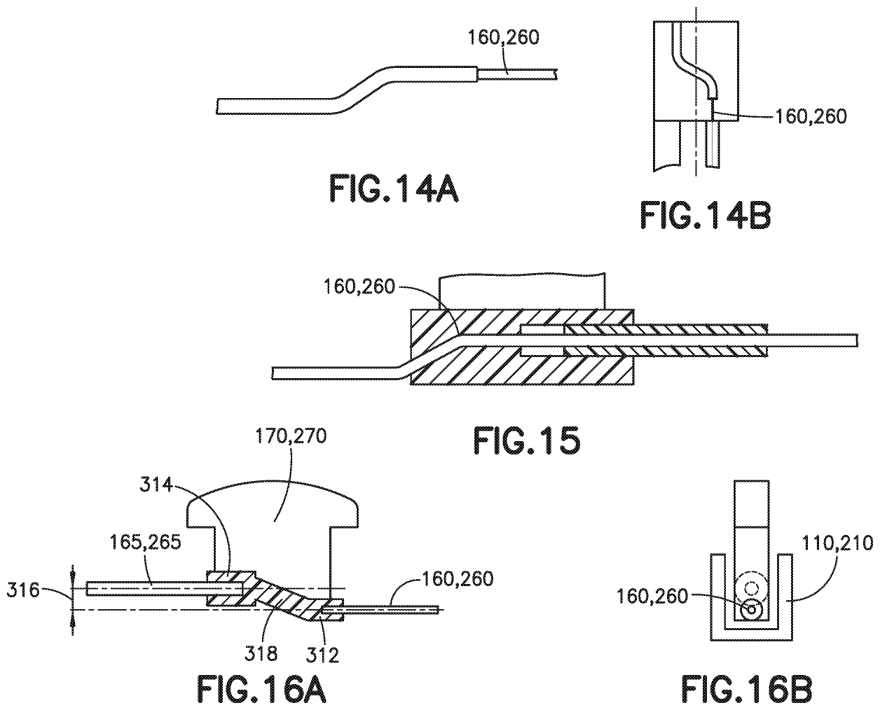

Referring to FIGS. 13-15, in one aspect or embodiment, a method of forming the catheter 160, 260 with the first portion 302 and the second portion 304 includes molding the catheter 160, 260 with a curved core pin pull to bend the catheter 160, 260 downward (FIG. 13) or pre-shaping the catheter 160, 260 to provide the first portion 302 and the second portion 304 (FIGS. 14A, 14B, and 15). In one embodiment, tubing may be placed on the mold core pin before being placed in the actuator mold. The polymer is then injected around the core pin and tubing to form the actuator. The core pin is subsequently pulled from the mold, leaving the actuator bonded to the tubing. In standard molding configurations, the core pin is straight so that it can easily be pulled from the mold, however, in certain embodiment of the present invention, the core pin is bent at an angle to hold the tubing in the desired shape during molding. The core pin then has to bend as it is pulled from the assembly. The core pin must be stiff enough to hold a specific shape in the mold, but then flexible enough to bend during removal. FIGS. 13-14 illustrate the curved core pin concept. Alternatively, as shown in FIG. 15, a straight core pin is shown, however, in this configuration the core pin is pulled from the mold at an angle with respect to the core pin on the other end to the angle the tubing downward.

Referring to FIGS. 16A-18, in one aspect or embodiment of the present application, the actuator 170, 270 of either device 100 or device 200, includes a catheter connection 312 connected to the catheter 160, 260 and a secondary catheter connection 314 connected to the secondary catheter 165, 265. The catheter 160, 270 and the secondary catheter 165, 265 each extend parallel to the longitudinal axis L1, L2 of the introducer 110, 210, with the catheter 160, 260 offset a distance 316 from the secondary catheter 165, 265.

Referring to FIGS. 16A and 16B, in one aspect or embodiment, the catheter connection 312 is offset from the secondary catheter connection 314 via an angled body portion 318. The offset of the catheter 160, 260 from the secondary catheter 165, 265 is in a direction perpendicular to the longitudinal axis L1, L2 of the introducer 110, 210, which positions the catheter 160, 260 towards the bottom of the introducer 110, 210 within the inner volume 113, 213 to minimize the degree to which the catheter 160, 260 may kink and/or buckle.

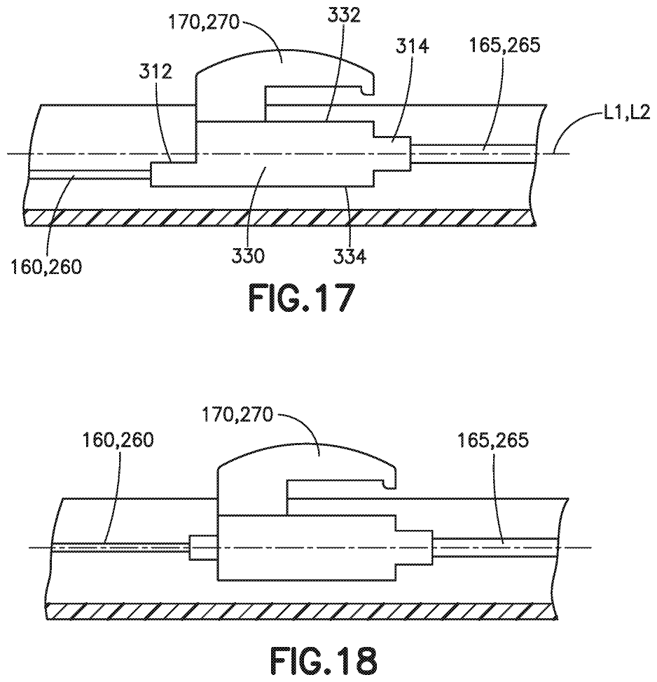

Referring to FIGS. 17 and 18, in one aspect or embodiment, the actuator 170, 270 includes a main body 330 having a top surface 332 and a bottom surface 334 positioned opposite from the top surface 332. The catheter connection 312 extends from the bottom surface 334 of the main body 330 of the actuator 170, 270. The secondary catheter connection 314 extends between the top surface 332 and the bottom surface 334 of the main body 330 of the actuator 170, 270.

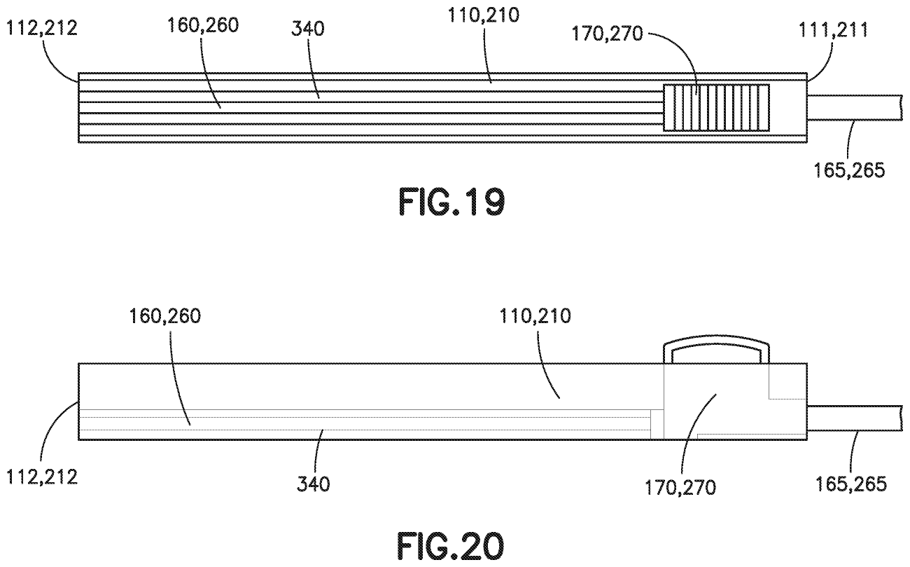

Referring to FIGS. 19 and 20, in some aspects or embodiments, the introducer 110, 210 of either device 100 or device 200, defines a groove 340 extending in a direction extending from the proximal end 111, 211 of the introducer 110, 210 to the distal end 112, 212 of the introducer 110, 210. The groove 340 is positioned within the inner volume 113, 213 of the introducer 110, 210 and configured to receive a portion of the catheter 160, 260 during use of the device 100, 200. The groove 340 may be U-shaped, V-shaped, or squared-shaped, although other suitable shapes may be utilized. The groove 340 is configured to support the catheter 160, 260 from three sides (left, right, and bottom) to further support the catheter 160, 260 during use to prevent kinking and/or buckling of the catheter 160, 260. The groove 340 may extend from the distal end portion 112, 212 of the introducer 110, 210 to the proximal end portion 111, 211, may extend from the distal end portion 112, 212 to a positioned intermediate the distal end portion 112, 212 and the proximal end portion 111, 211 of the introducer 110, 210, or may extend between the distal end portion 112, 212 and the proximal end portion 111, 211 of the introducer 110, 210.

In some aspects or embodiments, the catheter 160, 260 is formed from a polyimide tube, although other suitable materials may be utilized. Further, in some aspects or embodiments, the catheter 160, 260 may be a guidewire, sensor, or other instrument that may be inserted into vasculature or a vascular access device.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. A blood draw device for use with a peripheral intravenous catheter (PIVC) comprising:
a catheter having a proximal end, a distal end, and a sidewall therebetween defining a lumen;
an introducer having a proximal end, a distal end, and a sidewall therebetween defining an inner volume configured to movably receive the catheter, the introducer defining a longitudinal axis; and
an actuator movably coupled to the introducer and connected to the catheter, the actuator having a first portion disposed outside of the introducer and a second portion disposed in the inner volume of the introducer, wherein the catheter has a first portion extending parallel to the longitudinal axis of the introducer and a second portion extending oblique to the longitudinal axis of the introducer.

2. The blood draw device of claim 1, wherein the actuator is configured to move relative to the introducer to move the catheter between a first position, in which the catheter is disposed within the introducer, and a second position, in which the distal end of the catheter is disposed beyond the distal end of the introducer.

3. The blood draw device of claim 1, further comprising a secondary catheter having a proximal end, a distal end, and a sidewall therebetween defining a lumen, the secondary catheter connected to the actuator.

4. The blood draw device of claim 3, wherein the proximal end of the secondary catheter comprises a coupler.

5. The blood draw device of claim 1, wherein the actuator comprises a main body and an extension portion, the main body of the actuator extends parallel to the longitudinal axis of the introducer, the extension portion of the actuator receives the catheter and extends oblique to the longitudinal axis of the introducer.

6. The blood draw device of claim 1, wherein the introducer defines a groove extending in a direction extending from the proximal end of the introducer to the distal end of the introducer, the groove positioned within the inner volume of the introducer and configured to receive a portion of the catheter during use of the blood draw device.

7. The blood draw device of claim 6, wherein the groove is at least one of U-shaped, V-shaped, and squared-shaped.

8. A blood draw device for use with a peripheral intravenous catheter (PIVC) comprising:
a catheter having a proximal end, a distal end, and a sidewall therebetween defining a lumen;
a secondary catheter having a proximal end, a distal end, and a sidewall therebetween defining a lumen;
an introducer having a proximal end, a distal end, and a sidewall therebetween defining an inner volume configured to movably receive the catheter, the introducer defining a longitudinal axis; and
an actuator movably coupled to the introducer, the actuator having a first portion disposed outside of the introducer and a second portion disposed in the inner volume of the introducer, the actuator comprising a catheter connection connected to the catheter and a secondary catheter connection connected to the secondary catheter, wherein the catheter and the secondary catheter each extend parallel to the longitudinal axis of the introducer, with the catheter offset a distance from the secondary catheter.

9. The blood draw device of claim 8, wherein the catheter connection is offset from the secondary catheter connection via an angled body portion.

10. The blood draw device of claim 8, wherein the actuator comprises a main body having a top surface and a bottom surface positioned opposite from the top surface, and wherein the catheter connection extends from the bottom surface of the main body of the actuator.

11. The blood draw device of claim 10, wherein the secondary catheter connection extends between the top surface and the bottom surface of the main body of the actuator.

12. The blood draw device of claim 8, wherein the actuator is configured to move relative to the introducer to move the catheter between a first position, in which the catheter is disposed within the introducer, and a second position, in which the distal end of the catheter is disposed beyond the distal end of the introducer.

13. The blood draw device of claim 12, wherein the introducer defines a groove extending in a direction extending from the proximal end of the introducer to the distal end of the introducer, the groove positioned within the inner volume of the introducer and configured to receive a portion of the catheter during use of the blood draw device.

14. The blood draw device of claim 13, wherein the groove is at least one of U-shaped, V-shaped, and squared-shaped.

15. The blood draw device of claim 8, wherein the catheter is offset downward from the secondary catheter.

16. The blood draw device of claim 8, wherein the catheter is offset upward from the secondary catheter.

17. The blood draw device of claim 8, wherein the catheter is offset from the side from the secondary catheter.

18. A method of forming a catheter for a blood draw device for use with a peripheral intravenous catheter (PIVC), the blood draw device comprising a catheter defining a lumen, an introducer defining an inner volume configured to movably receive the catheter, the introducer defining a longitudinal axis, and an actuator movably coupled to the introducer and connected to the catheter, the method comprising:
molding the catheter to include a first portion extending parallel to the longitudinal axis of the introducer and a second portion extending oblique to the longitudinal axis of the introducer.

19. The method of claim 18, wherein molding the catheter comprises a curved core pin pull.

20. The method of claim 18, wherein molding the catheter comprises bending the second portion of the catheter relative to the first portion of the catheter.
